(19) United States
(12) Patent Application Publication
(10) Pub. No.: US 2012/0196284 A1
(43) Pub. Date: Aug. 2, 2012

(54) Method of Measuring Telomere Length

(75) Inventors: Abraham Aviv, Essex Fells, NJ (US); Masayuki Kimura, Edgewater, NJ (US)

(73) Assignee: UNIVERSITY OF MEDICINE AND DENTISTRY OF NEW JERSEY, Somerset, NJ (US)

(21) Appl. No.: 13/328,839

(22) Filed: Dec. 16, 2011

Related U.S. Application Data
(60) Provisional application No. 61/423,954, filed on Dec. 16, 2010.
(60) Provisional application No. 61/468,428, filed on Mar. 28, 2011.

(51) Int. Cl. C12Q 1/68

(52) U.S. Cl. 435/6.11; 435/6.12

(57) ABSTRACT

Disclosed herein is a novel method of measuring telomere length comprising determining the DNA content (Dx) of a sample, determining the telomeric content (T) of a sample, and determining the value of T/Dx.

### CROSS-REFERENCE TO RELATED APPLICATIONS

This application claims priority to U.S. Provisional Application 61/423,954 filed Dec. 16, 2010 and U.S. Provisional Application 61/468,428 filed Mar. 28, 2011, the disclosures of which are each incorporated by reference in their entireties.

### STATEMENT REGARDING FEDERALLY SPONSORED RESEARCH OR DEVELOPMENT

This invention was made with government support under grant numbers AG030678 and AG20132 awarded by the National Institutes of Health. Accordingly, the federal government may have certain rights in this invention.

### BACKGROUND OF THE INVENTION

Telomeres are repetitive DNA regions at the end of chromosomes that are essential for maintaining a stable genome. In humans and other mammals, telomeres comprise many kilobases of tandem TTAGGG repeats. Telomeres protect the ends of chromosomes and play a key role in cellular aging and senescence. Accordingly, telomeres have been closely studied to identify their relationship with aging and age-related disease. While the degree of causality between telomere length and certain disease states is still a topic of much research, it is generally agreed that telomeres are important biomarkers for a host of human diseases, including cancer, atherosclerosis, and possibly longevity.

Present methods to measure telomere length include Southern blot analysis of the terminal restriction fragments (TRFs), quantitative PCR (qPCR), and fluorescent in situ hybridization (e.g., Flow-FISH). These methods are fraught with shortcomings that have contributed to a growing debate about their research applications, diminishing their usefulness in clinical settings. For epidemiological research, Southern blots and qPCR methods are typically preferred over Flow-FISH, as Flow-FISH requires intact nuclei and samples must be processed within a short amount of time. While the Southern blot and qPCR methods have been extensively used in large-scale studies, they too display considerable problems that limit their practical use.

The Southern blot analysis of TRFs, which is currently considered the gold standard of telomere length measurement, provides the entire distribution of telomere lengths in the DNA sample. Southern blots also have a relatively low inter-assay coefficient of variation (<2%), and their measurements are expressed in absolute values (kb). However, Southern blots require significant amounts of DNA (˜3 μg per assay), are labor intensive, and are costly. They also require a considerable degree of expertise. TRFs include not only the canonical component of telomeres (i.e. the TTAGGG tandem repeats) but also their non-canonical component up to the nearest restriction site of the enzymes used to cut the DNA, which confounds the absolute telomeric length estimates achieved using the Southern blot method. Of importance, DNA integrity is essential for obtaining reliable results utilizing the TRF analysis.

The qPCR method to measure telomere DNA content provides the ratio of the telomeric product (T) normalized for a single-copy gene (S) and it measures the mean of the canonical component of telomeres. The method is high throughput, relatively inexpensive and requires little DNA (˜30-50 ng per assay). But a major disadvantage is that the PCR could amplify any measurement error, and questions have been raised regarding qPCR's reliability as compared with Southern blots. Further, the qPCR method only provides the average telomere length.

Telomere DNA content has also been measured by normalizing for alphoid centromere repeats. This method is not without its drawbacks, however. The length of alphoid centromeric repeats is highly variable among individuals. Moreover, in those cases in which this method has been employed, the measurements of telomeric content and centromeric content were performed either in duplicate blots (one for telomeric content and the other for centromeric content), or in the same blot in which the hybridization with the telomeric probe was followed by stripping the probe and re-hybridizing with the centromeric probe. These measurement methods introduce major confounders that could increase measurement error; for example, stripping the membranes and re-probing may raise background signal, resulting in an increase in the measurement error.

With regard to dot blot analysis, when DNA content is measured in solution while the telomere DNA is quantified on the blot, at least three intrinsic errors are present. First, no matter how accurate the measurement in solution might be, the measurement of the DNA itself has its own intrinsic error, including a potential shift in the DNA standard on different runs, which would increase the inter-assay variation. Second, small amounts of DNA might adhere to the walls of tubes, pipette tips and the blot apparatus, such that the amount of DNA actually in the dot is not the amount that is presumed to be pipetted onto it. Third, when pipetting into the dot, no matter how accurate the pipetting might be, the DNA input still varies for different dots. Together, these 3 tiers of error exert a considerable confounder on the results.

Clearly, there is an escalating need for a simple, reliable, and high throughput method to accurately measure telomere length for research and clinical purposes. This need will only increase with the anticipated expansions of research and clinical applications that will require telomere length measurements.

### SUMMARY OF THE INVENTION

The present invention avoids the errors present in the art by taking advantage of the unique feature of the SYBR Dx DNA Blot Stain to measure the total DNA content in the dot without interference with the hybridization of the telomere probe. Such feature enables measuring the relative amount of telomere DNA content to total DNA in the dot itself regardless of the extent of the error in the input DNA.

Described herein is a method of measuring telomeric DNA content (T). The method involves the use of blots, such as dot or slot blots, in which DNA content (Dx) in a sample is measured by a DNA blot stain. The blot is then hybridized with a telomeric probe, and T is normalized for Dx. Thus, T/Dx provides a measure of telomeric DNA content in a sample, and thus a way of measuring telomere length. The method requires minimal DNA (˜20 ng/assay), is simple to use, has a relatively low inter-assay coefficient of variation (<6%), and can be adopted for high throughput analysis. Because the method is not PCR-based, the potential for measurement error is reduced in comparison to other PCR-based methods.

It is an object of this invention to provide a method of measuring telomere length comprising the steps of (a) determining the DNA content (Dx) of a sample; (b) determining the telomeric content (T) of a sample, and (c) determining the value of T/Dx.

In certain embodiments, the sample is applied to a membrane. In further embodiments, the membrane comprises nylon.

In certain embodiments, the determination of Dx is performed by applying to the sample a composition capable of visualizing the total nucleic acid in the sample. In some embodiments, the composition is a DNA blot stain. In further embodiments, the composition is SYBR DX DNA blot stain.

In certain embodiments, the determination of T is performed by hybridizing the sample with a labeled telomeric probe. In some embodiments, the labeled telomeric probe comprises the DNA sequence CCCTAA. In some embodiments, the probe is labeled with a non-radioactive label. In other embodiments, the probe is labeled with a radioactive label. In further embodiments, the probe is labeled with digoxigenin.

In certain embodiments, the sample comprises DNA isolated from leukocytes. In other embodiments, the sample comprises DNA isolated from other cells, organs or tissues.

It is a further object of this invention to provide a kit for measuring telomere length comprising DNA blot stain, labeled telomere probe; and one or more membranes. In some embodiments, the kit further comprises at least one buffer. In further embodiments, the kit comprises instructions for utilizing the kit for practicing the method disclosed herein. In some embodiments, the kit comprises a composition capable of detecting the labeled telomere probe. In certain embodiments, the kit comprises CDP-Star solution, DNase-free water, or standard DNA. In further embodiments, the kit comprises denaturing buffer, neutralizing buffer, saline-sodium citrate buffer (SSC), tris-borate-EDTA buffer (TBE), hybridization buffer, washing buffer, maleic acid buffer, blocking buffer, or detection buffer. In some embodiments, the DNA blot stain is SYBR DX DNA blot stain. In some embodiments, the composition capable of detecting the labeled telomere probe is anti-DIG alkaline phosphatase antibody conjugate.

It is a further object of this invention to provide a kit for measuring telomere length comprising SYBR DX DNA blot stain, DIG-labeled telomere probe, anti-DIG-alkaline phosphatase antibody conjugate, one or more types of nylon membranes, denaturing buffer, neutralizing buffer, saline-sodium citrate buffer (SSC), tris-borate-EDTA buffer (TBE), hybridization buffer, washing buffer, maleic acid buffer, blocking buffer, detection buffer, CDP-Star solution, DNase-free water, and standard DNA.

### BRIEF DESCRIPTION OF THE DRAWINGS

FIG. 1 depicts dot blot staining of SYBR Dx and the telomere (TTAGGG) signal. FIG. 1(a) displays the SYBR Dx dot blot staining of different samples in triplicate. Samples used for the DNA standard are at the right lower corner. FIG. 1(b) displays the linearity of the staining between 5-35 ng of standard DNA. Each data point is the mean of triplicate measurements. FIG. 1(c) shows dot blots of the telomere signal, while FIG. 1(d) shows the linearity of the telomere signal within the 5-35 ng of standard DNA. Each data point is the mean of triplicate measurements. Vertical bars denote SD. No vertical bar indicates that the SD is within the space of the data point.

FIG. 2 depicts the relationship between mean telomere length, measured by Southern blots of the TRFs, expressed in kilobases (kb), versus the ratio of T (telomere amount)/Dx (DNA amount), measured by dot blots, and versus the ratio of T (telomere product)/S (single gene product), measured by qPCR. FIGS. 2(a) and 2(b) show the TRF products generated by HinfI/RsaI and by HphI/MnlI versus the T/Dx. FIGS. 2(c) and 2(d) show the TRF product generated by HinfI/RsaI and by HphI/MnlI versus T/S.

FIG. 3 depicts the relationship between mean telomere length, measured by Southern blots of the TRFs, generated by HinfI/RsaI, versus the ratio of T (telomere amount) and Dx (DNA amount), measured by dot blots. Data displayed in this figure are a composite of data displayed in FIG. 2 plus an additional set of measurements in leukocytes from 7 newborns and 7 elderly persons (aged 90-96 years old).

FIG. 4 depicts the effect of DNA sonication on the TRF and the T/Dx measurements. For all panels, lane 1=no sonication; lane 2=0.2 sec×5 pulse (sonicator set to position 1.5); lane 3=0.2 sec×5 pulse (sonicator set to position 2); lane 4=0.2 sec×10 pulse (sonicator set to position 2). FIG. 4(a) illustrates DNA integrity (arrow indicates the DNA crown). FIG. 2(b) illustrates TRF length distribution. FIG. 2(c) shows T/Dx results based on 3 DNA samples.

### DETAILED DESCRIPTION OF THE INVENTION

The method of the invention is based on the premise that the amount of DNA in nuclei of somatic cells is constant. Thus, the ratio of telomere repeats (T) to total DNA content (Dx), i.e. T/Dx, provides a measure of mean telomere content in a sample. This conclusion is reasonable provided that the amount of the repeats is within the discernible range of the method and the DNA does not contain large amounts of heterogeneous telomere-like sequences within its bulk, as is apparently the case for human DNA and other mammals.

The DNA content doubles in cells undergoing mitosis, but so does the amount of telomere repeats. Therefore, the T/Dx ratio is still constant in mitotic cells. In addition, females have two X chromosomes, while men have only one X chromosome and the much smaller Y chromosome. In principle, the nuclei of females should have more DNA, but evidently this effect is small, amounting to a difference of ˜1.6%. If future large-scale studies show that the sex difference in DNA content impacts the T/Dx ratio in relation to indices of interest, an adjustment factor can be introduced to account for this sex difference, or standard curves can be developed separately for women and men.

Generally, the method of the invention includes the following step: First, a sample is applied to a membrane for analysis, such as those typically used in a DNA dot and blots (including, without limitation, nylon membranes such as Zeta-Probe® (BioRad), Hybond™-N and Hybond™-N+ (Amersham). Second, the DNA content (Dx) of the sample on the blot is measured. Such measurement can be achieved, for example, by applying a composition capable of quantifying, detecting and/or visualizing total DNA in the sample, such as SYBR DX DNA Blot Stain (S-7550) (Invitrogen), that will not interfere with subsequent hybridization steps. Third, the blot is hybridized with a labeled telomeric probe, and the telomeric content (T) is measured. Such measurement can be achieved, for example, by applying a labeled telomeric probe to the blot (available at Roche). Such labels can include, for example, digoxigenin, other non-radioactive labels, radioactive labels, or any other suitable labels. The measurement can be taken by utilizing a composition capable of detecting the label, such as, for example, anti-DIG-alkaline phosphatase antibody conjugate. Finally, T/Dx is determined, which is indicative of the mean telomere content normalized for the DNA content in the sample.

This approach has several advantages over the previous methods: the staining with a DNA stain, such as SYBR DX DNA Blot Stain, which detects single-stranded DNA on the transfer membranes (blots), does not interfere with the telomeric probe hybridization; the detections of both DNA and telomeric signals are performed within their linear ranges; the measurement of DNA content can be completed within one hour; and small quantities (˜20 ng and as little as 5 ng) of DNA are used per dot (compared with 2.5 μg of the Southern blots). The method can be automated for high throughput analysis, but does not rely on PCR to obtain the telomeric (and reference gene) signals. Moderate DNA degradation has no effect on the T/Dx ratio.

Like the qPCR method, the telomere content measured by using the blot analysis captures only the canonical region (i.e., only the TTAGGG repeats) of the telomeres. When the linear line of TRF length is regressed to T/Dx=0, it is routinely observed that the so-called sub-telomeric (non-canonical) region is <2 kb. In contrast, based on the qPCR analysis of telomere repeats content, regression of TRF length to T/S=0 yields sub-telomeric value>3 kb and often >4 kb. That said, it is not known whether the linearity of the TRF length vs. T/Dx, or TRF vs. T/S, is maintained at the lower boundary of telomere length that is not captured by the data. It has long been suspected that the previously observed sub-telomeric values were too high. Using the inventive method, the non-canonical values have been observed to be significantly smaller, typically <2 kb. It is believed that the large values observed with the qPCR methods further illustrate the inherent error present in this method.

The methods of the present invention could be particularly important in determining leukocyte telomere length (LTL). The evidence is strong that LTL is associated with aging-related diseases, principally vascular aging, as expressed in atherosclerosis, and survival in the elderly. LTLs may also be linked to longevity. Patients with atherosclerosis display a shorter LTL than their peers who lack the clinical manifestations of the disease. Relatively short LTLs are also observed in connection with clinical circumstances that heighten the risk for atherosclerosis, including obesity, insulin resistance, sedentary lifestyle and smoking. In addition, recent studies, including research in same-sex elderly twins, clearly show that short LTL in the elderly is associated with diminished survival. Because atherosclerosis is the main cause of death in the elderly, it might also contribute to their death from infection, diabetes, frailty, and/or dementia. Therefore, short LTL might be linked to human longevity through atherosclerosis. Considering the significance of atherosclerosis as a medical problem, tracking LTL dynamics in individual patients could be used in clinical settings for disease prevention and therapy. Simple and better methods for measuring LTL, such as the method disclosed herein, will enable researchers and clinicians to gain vital information above and beyond conventional biomarkers about persons at risk for atherosclerosis and premature death. Such information will be particularly relevant if short LTL is diagnosed at a relatively young age, prior to manifestations of conventional risk factors and disease onset, so that appropriate preventative measures can be taken.

For ease of practicing this method, kits containing certain required components may be distributed or marketed to researchers and clinicians. The components of the kits may include, without limitation: DNA blot stain; labeled telomere probe and a composition capable of detecting the same; one or more membranes; buffers; CDP-Star solution DNase-free water; and standard DNA. The DNA blot stain can be, for example, SYBR DX DNA blot stain. The buffers can include one more of the following buffers: denaturing buffer, neutralizing buffer, saline-sodium citrate buffer, tris-borate-EDTA buffer, hybridization buffer, washing buffer, maleic acid buffer, blocking buffer, and detection buffer. The telomere probe label can be, for example, digoxigenin (DIG), and the composition for detecting it can be, for example, anti-DIG-alkaline phosphatase antibody conjugate. The membrane material can, for example, comprise nylon.

### EXAMPLES

General procedures: DNA was isolated by Gentra Puregene Blood kit (Qiagen) from leukocytes donated by 28 individuals. As LTLs in this group did not encompass the overall spectrum of LTLs seen throughout the entire human lifespan, in a second set of experiments the LTLs in leukocyte samples from 7 newborns and 7 elderly persons (aged 90-96 years) was also determined. Measurements of mean telomere length by Southern blots of the TRFs were performed in duplicate after digesting the DNA with HinfI and RsaI restriction enzymes. The telomere probe used for both the TRF length analysis and dot blot analysis consisted of three CCCTAA oligonucleotide repeats, which are complementary to the canonical TTAGGG sequence in humans, and it is labeled at the 3′ end with digoxigenin (DIG).

Telomere repeats/DNA ratio by dot blot analysis: The linear range of the SYBR DX stain (5-35 ng) was first established. The corresponding telomere signals also displayed linearity within this DNA range.

The assays were performed as follows: Each DNA aliquot (3.3 μL; 20 ng/μL, measured by UV for each samples, 5, 15, 25, 35 ng/μL for the DNA standard) was diluted into 16.5 μL of denaturing solution (0.5 M NaOH, 1.5 M NaCl) and incubated at 55° C. for 30 min. Neutralizing solution (495 μL; 0.5 M Tris-HCl, 1.5 M NaCl) was added. A positively charged nylon membrane (Roche) was soaked in distilled water for 10 min and Bio-Dot Microfiltration Apparatus was assembled according to the manufacturer's instructions. Each well was washed once with 200 μL of water. The 156 μL of neutralized sample or standard was loaded into each well (in triplicate) and subjected to gentle vacuum. Thereafter, each well was washed once with 200 μL. 2× saline-sodium citrate buffer (SSC), the membrane was removed, rinsed in 2×SSC and UV-cross linked.

For DNA Blot Staining, the membranes were washed in distilled water for 10 min, rinsed with 0.5× Tris-borate-EDTA buffer (TBE) and stained with 5 mL SYBR DX stain (1000× dilution in 0.5×TBE) for 30 min. The fluorescence signal was measured by Typhoon 9400 (GE Healthcare). The DNA amount of each sample was calculated based on the known standards.

The membrane was pre-hybridized in 5×SSC, 0.1% Sarkosyl, 0.02% Sodium Dodecyl Sulfate (SDS), and 1% blocking reagent (Roche) at 65° C. for 2 hours and hybridized at 65 ° C. with the telomeric probe overnight in the same solution. The membrane was washed three times at room temperature in 2×SSC and 0.1% SDS each for 15 min and once in 2×SSC for 15 min. The DIG-labeled probe was detected by the DIG luminescent detection procedure (Roche) and exposed on x-ray film. The amount of telomere repeats from each sample was calculated from the standard.

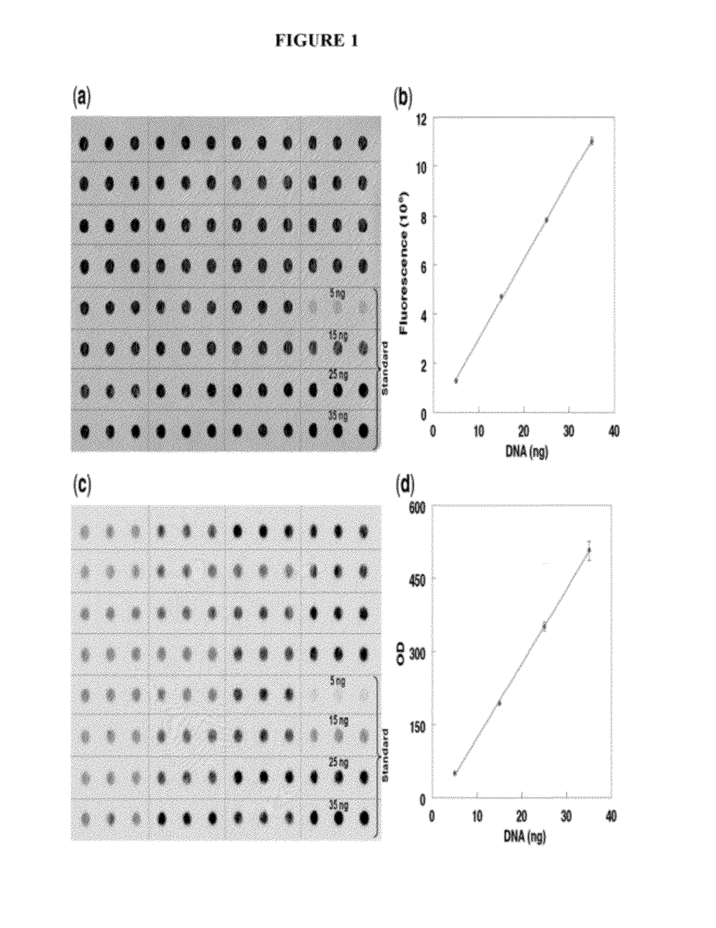

Using ImageQuant, the total intensity above local background surrounding each dot was independently determined for each of the 3 dots in each rectangle show in FIGS. 1(a) and 1(c). The mean value of the three dots was used for the standard curves displayed in FIGS. 1(b) and 1(d). For each sample, the T/Dx for each of the 3 dots was independently obtained and the means of the 3 T/Dx values was derived.

Telomere Length/DNA content measurements by Southern blot analysis of the TRFs and by qPCR: The mean TRF length using either HinfI/RsaI or HphI/MnlI, was obtained. HinfI/RsaI, which typically is used in TRF length analysis in most laboratories, cuts the DNA within the non-canonical sub-telomeric region. However, HphI/MnlI cuts the DNA at telomere repeat variants that are more proximal regions of the telomeres. Therefore, digestion with HinfI/RsaI usually results in a mean TRF length that is longer by ˜1 kb than that resulting from HphI and MnlI digestion.

Measurements of the mean TRF length were performed. Briefly, DNA integrity was first evaluated by SYBR Green I, after resolving each sample (10 ng) on 1% agarose gel at 200V for 60 minutes. Thereafter, samples were digested with restriction enzymes HinfI (10 U) and RsaI (10 U; Roche) or HphI (3.1 U)/MnlI (3.1 U) (New England Biolabs, Ipswich, Mass.). DNA samples (3 μg each), and DNA ladders (1 kb DNA ladder plus λ DNA/Hind III fragments; Invitrogen, Carlsbad, Calif.) were resolved on 0.5% agarose gels for most subjects and on a 0.6% agaorse gel for subjects 90-96 years (20 cm×20 cm) at 50V (GNA-200 Pharmacia Biotech). After 16 hours, the DNA was depurinated for 15 minutes in 0.25 N HCl, denatured 30 minutes in 0.5 M NaOH/1.5 mol/L NaCl and neutralized for 30 minutes in 0.5 mol/L Tris, pH 8/1.5 M Nacl. The DNA was transferred for 1 hour to a positively charged nylon membrane (Roche) using a vacuum blotter (Boeckel Scientific, Feasterville, Pa.). Thereafter, membranes were hybridized at 65° C. with the DIG-labeled telomeric probe overnight in 5×SSC, 0.1% Sarkosyl, 0.02% SDS, and 1% blocking reagent (Roche). The membranes were washed three times at room temperature in 2×SSC, 0.1% SDS each for 15 minutes and once in 2×SSC for 15 minutes. The DIG-labeled probe was detected by the DIG luminescent (Roche) and exposed on X-ray file. All autoradiographs were scanned, and the TRF signal was digitized. The optical density values versus DNA migration distances were converted to optical density (adjusted for background)/molecular weight versus molecular weight.

The measurement of telomere repeats by qPCR provides the ratio of the telomeric product (T) normalized for a single-copy gene product (S). This measurement was performed using minor modifications of the original method and beta globin as S.

DNA donors provided written consent. The study was approved by the Institutional Review Board of University of Medicine and Dentistry of New Jersey, New Jersey Medical School.

Results: FIG. 1 displays dot blot staining of SYBR DX and the telomere (TTAGGG) signal for triplicate samples (20 ng/sample), including the DNA standard samples. Clearly, at the range of 5-35 ng both the SYBR DX dye and the telomere signal are highly linear. The intra-assay coefficient of variations (%) of triplicates samples (analyzed at the same time) were: T=4.4. Dx=2.6, T/Dx=5.4 (N=56). The inter-assay coefficient of variation of duplicate samples (analyzed on different days) of T/Dx was T=5.7 (N=28).

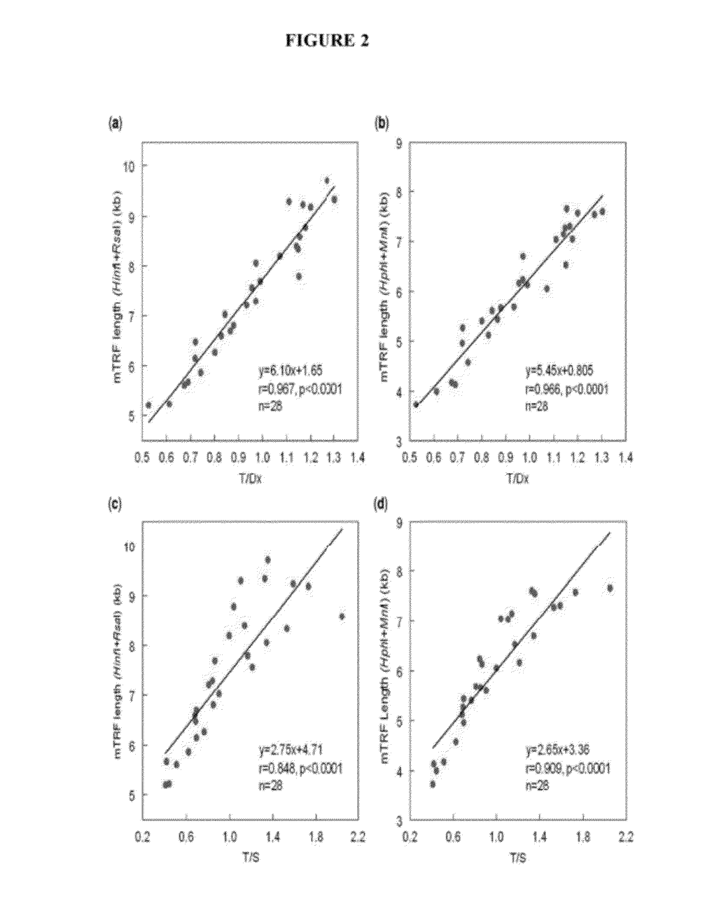

FIG. 2 displays the relation between T/Dx and T/S with the mean TRF length generated by either HinfI/RsaI or HphI/MnlI. For the T/Dx (2(a) and 2(b)), strong correlations are observed with mean TRF length regardless of the restriction enzymes used to generate the TRFs. Strong correlations are also observed for the relation of T/S (2(c) and 2(d)) with the mean TRF length, although the correlations are not as robust as those between T/Dx and the mean TRF length, particularly the HinfI/RsaI product.

Both the present dot blot analysis and the qPCR-based methods measure only the canonical part of the telomeres, which consist of strictly TTAGGG repeats. In contrast, the TRF length generated by the Southern blots includes both the canonical and non-canonical region extending to the nearest restriction site. Estimates of the extrapolated non-canonical regions can be obtained from the regressions displayed in FIG. 2 when T/Dx=0 or T/S=0, with the stipulation that linearity of the regressions is maintained beyond the empirical data. Accordingly, for TRFs generated by HinfI/RsaI, when T/Dx=0, mean TRF=1.65 kb; for TRFs generated by HphI/MnlI, when T/Dx=0, mean TRF=0.805 kb. However, for TRFs generated by HinfI/RsaI, when T/S=0, mean TRF=4.71 kb; for TRFs generated by HphI/MnlI, when T/S=0, mean TRF=3.36 kb. These differences in the extrapolated lengths of the non-canonical segment of the TRFs probably stem from deviation from linearity of relation between T/S versus mean TRF length (FIGS. 2(c) and 2(d)), which is already observed for the empirical data of the regressions displayed in FIGS. 2(c) and (d). This finding has been shown by others. The underlying causes are not certain but it is presumed that they relate to problems with the PCR of S, T or both.

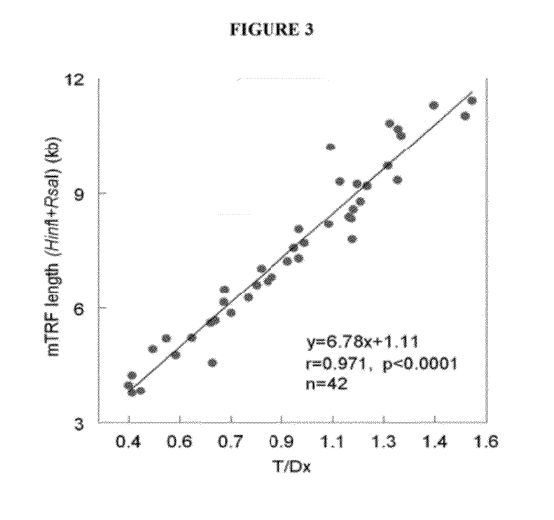

The data displayed in FIG. 2 do not cover the entire spectrum of LTL seen throughout the entire human lifespan. For this reason, in a second set of experiments, LTL was measured (by dot blot analysis and Southern blots of the TRFs generated by HinfI/RsaI in leukocytes from newborns and exceptionally old persons. FIG. 3 consists of data derived from results generated in the first set of experiments (shown in FIG. 2(a)) and the second set of experiments. The linear relation between the mean TRF and the T/Dx is maintained in leukocytes from donors whose age range essentially covers the entire human lifespan. Each of the data points described in FIGS. 2 and 3 represents (i) the mean of two different runs for the mean TRF length (one measure/run); (ii) the mean of two T/Dx runs (three measures/run); and (iii) the mean of three T/S runs (three measures/run). The coefficient of variations for the intra-assay and inter-assay measurements were computed as the ratios of the standard deviation (DV) to the mean of measurements performed on the same run and the mean of measurements performed on different runs, respectively.

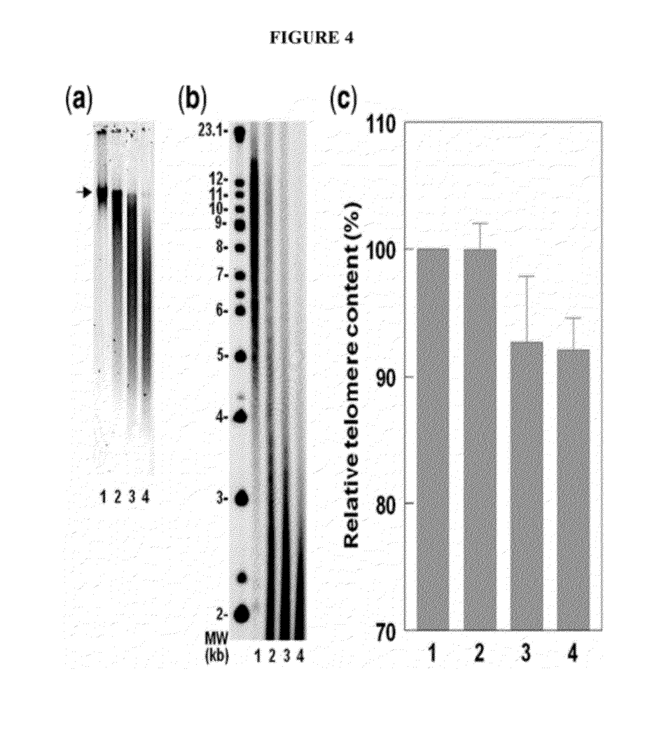

The effect of DNA degradation on the TRF length and the T/Dx measurement is shown in FIG. 4. DNA was degraded by sonication (Ultrasonic Processor XL2020, microtip probe) and its integrity determined (using SYBR Green I) by resolving 10 ng of DNA on 1% (wt/vol) agarose gel at 200 V for 60 minutes (see FIG. 4(a)). Moderate DNA degradation, where the DNA crown is still compact but a long tail of DNA smear is observed, already compromises any meaningful analysis of the TRFs. This is because the TRFs are considerably shortened, so that the TRF smear extends to the edge of the gel (see FIG. 4(b)), outside the routine scanning region of the TRFs, which rarely extends lower than 1.2 kb. More degradation further exacerbates this effect. In contrast, the T/Dx results are not modified by moderate DNA degradation (see FIG. 4(c)). However, more severe DNA degradation caused 7-8% decline in the T/Dx values, probably because the stretches of telomere repeats become too short for an effective annealing of the telomere probe.

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those show and described herein will become apparent to those skilled in the art and are intended to fall within the scope of the appended claims.

What is claimed:

1. A method of measuring telomere length comprising the steps of:
(a) determining the DNA content (Dx) of a sample;
(b) determining the telomeric content (T) of a sample; and
(c) determining the value of T/Dx.

2. The method of claim 1, wherein the sample has been applied to a nylon membrane.

3. The method of claim 1, wherein the determination of Dx is performed by applying to the sample a composition capable of visualizing the total nucleic acid in the sample.

4. The method of claim 3, wherein the composition is a DNA blot stain.

5. The method of claim 3, wherein the composition is SYBR DX DNA blot stain.

6. The method of claim 1, wherein the determination of T is performed by hybridizing the sample with a labeled telomeric probe.

7. The method of claim 6, wherein the labeled telomeric probe comprises the DNA sequence CCCTAA.

8. The method of claim 6, wherein the probe is labeled with digoxigenin.

9. The method of claim 1, wherein the sample comprises DNA isolated from leukocytes.

10. A kit for measuring telomere length comprising:
(a) DNA blot stain;
(b) labeled telomere probe; and
(c) one or more membranes.

11. The kit of claim 10, further comprising at least one buffer.

12. The kit of claim 10, further comprising instructions for utilizing the kit for practicing the method disclosed herein.

13. The kit of claim 10, further comprising a composition capable of detecting the labeled telomere probe.

14. The kit of claim 10, further comprising CDP-Star solution, DNase-free water, and standard DNA.

15. A kit for measuring telomere length comprising:
(a) SYBR DX DNA blot stain;
(b) DIG-labeled telomere probe;
(c) anti-DIG-alkaline phosphatase antibody conjugate;
(d) one or more nylon membranes.
(e) denaturing buffer, neutralizing buffer, saline-sodium citrate buffer (SSC), tris-borate-EDTA buffer (TBE), hybridization buffer, washing buffer, maleic acid buffer, blocking buffer, and detection buffer;
(f) CDP-Star solution;
(g) DNase-free water; and
(h) standard DNA.
